(19) United States
(12) Patent Application Publication
(10) Pub. No.: US 2009/0010937 A1
(43) Pub. Date: Jan. 8, 2009

(54) Membrane Attack Complexes Associated with Circulating Immune Complexes

(76) Inventor: Anil K. Chauhan, Ballwin, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/587,819

(22) PCT Filed: Jan. 28, 2005
(86) PCT No.: PCT/US05/02586

§ 371 Date: Jul. 30, 2008

Related U.S. Application Data
(60) Provisional application No. 60/539,705, filed on Jan. 28, 2004.

(51) Int. Cl. G01N 33/564; C07K 14/47; A61K 39/395; A61P 37/00; A61K 38/02

(52) U.S. Cl. 424/141.1; 530/350; 436/507; 435/7.92

(57) ABSTRACT

The invention relates to demonstrate the presence of non covalently linked complement proteins with CIC. Invention demonstrate the presence of C5 and MAC on CIC. The invention provides methods to measure M complement proteins C1q, C3, C4, C5 bound to CIC as markers of disease activity and pathogenicity for complement and CIC mediated diseases. The invention also relates to modifications and blocking of formation of MAC on the CIC by developing a useful reagent that can be monoclonal antibody, active molecule, mimotope or peptide molecule. The invention describes usefulness to reduce the non-covalent complement components, formation of MAC and association of C5 to CIC for therapeutic application in pathological disease with involvement of CIC and complement.

### CROSS-REFERENCE TO RELATED APPLICATIONS

This application claims priority from U.S. application 60/______ in, filed Jan. 28, 2004, which is fully incorporated herein by reference.

### STATEMENT REGARDING FEDERALLY SPONSORED RESEARCH OR DEVELOPMENT

Not applicable.

### REFERENCE TO A SEQUENCE LISTING

Not applicable.

### BACKGROUND OF THE INVENTION

Complement pathway is made of more than 35 plasma proteins that play a key role in host defense against microbial infections and continuous clearance of apoptotic debris. The formation of an antigen-antibody complex (also referred to as immune complex) is the principal mechanism of complement activation. Complement plays a key role in the antigen presentation and regulation of antibody responses by B cells. The activation of complement proteins primarily occurs by three major pathways—classical, alternate and mannose binding lectin pathways. Each pathway leads to formation of C3 convertase which then cleaves the C3 complement protein to generate C3a, an anaphylotoxin and a second subunit, C3b, that participates in formation of C5 convertase. At the C5 convertase level the three complement pathways converge leading to cleavage of the C5 molecule that results in formation of C5b and C5a, another potent anaphylotoxin. The C5b complement protein forms the substrate for formation of a macromolecular complex by associating with the complement proteins C6, C7, C8, and up to sixteen molecules of C9, to form the terminal complement complex (TCC), also called a membrane attack complex (MAC), or simply, C5b-9. The MAC formation occurs once the complement protein C5 is cleaved into C5a and C5b. It is known that once the C5 convertase splits C5 into C5a and C5b, C5b then associates with C6 and C7 to form C5b-7, which complex then inserts into the cell membrane. Once the C5b-7 becomes inserted into the cell wall, it then acts as substrate for addition of the remaining components of MAC complex namely C8 and C9 on the cell membrane. The activation of complement pathway by CIC (circulating immune complexes) subsequently leads to the formation of a high number of MAC molecules on CIC. These CICs transfers high doses of MAC to cell membrane thus inflicting reversible cell damage leading to cell lysis. The amount of MAC transferred to cell membrane determines whether the cell will be destined for apoptosis or necrosis. Sublytic doses of MAC on cell walls triggers multiple signaling pathways initiating pleiotropic responses.

Complement plays a key role in regulating the innate and adoptive immune responses. The activation of complement pathway and subsequent generation of C5a and MAC (TCC, C5b-9) by polymerization of terminal components of the complement are the key mediators of complement induced signaling pathways and inflammatory responses. In the present invention, for the first time applicant demonstrates the presence of C5 and MAC complexes on the CIC and demonstrates that the CIC act as the substrate for the formation of MAC and their subsequent transfer to the cell membrane. Applicant also shows that the complement activation products C1q, C3, C4, C5 and C5b-9 present on CIC are non-covalently linked to CIC. Applicant also demonstrates that CIC carrying higher levels of complement are present in the serum of patients during disease activity, e.g., for rheumatoid arthritis, systemic lupus erythematosus, Chemokine production by alveolar macrophages in the presence of C5a or C5b-9 [Czermark et al, Am. J. Pathol. 154(5):1513 (1999)] is significantly higher after treatment with circulating immune complexes (CIC) composed with IgG isotype of immunoglobulin. It was noted that for production of macrophage inflammatory protein-2 (MIP-2), cytokine-induced neutrophil chemoattractant (CINC), monocyte chemoattractant protein-1 (MCP-1) and macrophage inflammatory protein-1α (MIP-1 α). by C5a and or C5b-9 in an intrapulmonary environment, the presence of IgG containing CIC was essential. [Czermak et al, Am. J. Pathol. 154(5):1513 (1999)] The data accumulated over years have supported the notion that the formation of C5 convertase leading to generation of C5a and C5b-9 is a major component of chronic inflammatory processes associated with atherosclerosis [Yasojima et al, Am. J. Pathol. 158(3):1039 (2001)], myocardial infarction and myocardial ischemia and reperfusion injury [Vaveka et al, Circulation 97:2259 (1998); Afanasyeva et al, Am. J. Pathol. 161(2):351 (2002)].

In view of the significance of the activation of the C5 molecule and subsequent generation of C5a and C5b-9, the mechanisms and localization of C5a and C5b-9 during activation are shown be applicant to be tools for mediating immune activity. The complement activation occurs via three main pathways that converge at the C5 convertase step. The C5 convertase activity to split the C5 molecule into C5a and C5b is provided by C3bBb3b, in the alternative pathway convertase and by C4b2aC3b, in the classical pathway convertase. In either case, the C5a fragment is released and the two-chain C5b fragment provides the substrate for the formation of the C5b-9. The larger fragment C5b associates with C6 and C7, which forms an amphiphilic entity, capable of inserting itself into the cell membrane. C8 then joins the complex and unwinds in the membrane. Finally up to sixteen molecules of C9 join to form C5b-9 complex.

It is known that the cleavage of C5 occurs in a fluid phase, thus generating C5b and C5a. The association of C5b to C6 and C7 occurs in the fluid phase leading to the formation of C5b-7 complex, which then inserts itself into the plasma membrane. The fate of soluble C5b-7 complex is determined by the soluble complement inactivating factors such as S protein, clusterin or vitronectin present in soluble phase. The binding of C5b-7 to these proteins inhibits the insertion of the complex into the cell membrane. In a rat alveolar macrophage model it was reported that soluble MAC, which has little ability to bind to cell surfaces, did not enhance lung injury after intrapulmonary deposition of IgG CIC [Czermak et al, Am. J. Pathol. 154(5):1513 (1999),].

Recent work on the C5b-9 molecular complex has shown the importance of the C5b-9 molecular complex in the apoptosis, necrosis and pro-inflammatory pathways [Bohana-Kashtan et al, Molecular Immunology 41:583 (2004)]. The C5b-9 complex is the principal mediator of injury induced by antibodies experimentally directed against glomerular cell membranes. C5b-9 in sublytic concentration enhances the production of endothelial intercellular adhesion molecule-1 (ICAM-1) and E selectin, while directly inducing production of interleukin 8 (IL-8) and monocyte chemo attractant protein-1 (MCP-1) [Kilgore et al, J. Immunol. 155:1434(1995)]. It also has been reported that the C5b-9 complex activates transcription factors nuclear factor-κ B (NF-κ B) and AP-1 resulting in the production of interleukin-6 (IL-6) and interleukin-8 in human smooth muscle cells [Viedt et al, FASEB J. 14:2370(2000].

In podocytes, the number of C5b-9 complexes inserted into cell membranes determines whether a cell undergoes necrosis. Formation of sublytic C5b-9 complex on the cell membrane results in release of calcium, activation of specific signaling pathways, and an increase in growth factor production, as well as increased oxidants and proteases [Couser W. G. J. Am. Soc. Nephrol. 1:13(1990); Cybulsky et al, Am J. Pathol. 155:1701(1999)]. The sublytic dose of C5b-9 complex on cell membrane activated cell cycle related genes, i.e. p53, p21, growth arrest DNA damage-45 (GADD45), checkpoint kinase-1 (CHK-1) and CHK-2. The extra cellular signal-regulated kinase (ERK) is involved in a critical pathway involved in regulating these cell cycle related proteins following C5b-9 induced DNA damage [Pippin et al, J. Clin. Invest. 111:877 (2003).

Assembly of C5b-9 on cells of the arterial wall induces cell lysis. The sublytic assembly of C5b-9 on smooth muscle cells and endothelial cells induce cell activation and proliferation. Sublytic assembly of C5b-9 on the plasma membrane activates p 38 MAPK, Janus kinase (JAK) 1, signal transducer and activator (STAT) 3 and STAT 4 in endothelial cells. [Niculescu et al, J. Immunol. 158:4405 (1997)].

In the passive Heymannn nephritis model of membranous nephropathy, the assembly of C5b-9 induces glomerular epithelial cell (GEC) injury and proteinuria that is partially mediated via production of eicosanoids. The sublytic formation of C5b-9 induces phosphorylation of epidermal growth factor receptor (EGF-R), fibroblast growth factor receptor-2 and hepatocyte growth factor receptor. The phosphorylation of tyrosine (204) of ERK-2 as well as free [(3) H] arachidonic acid (AA) and prostaglandin E (2) was stimulated by the formation of C5b-9 on cell membrane. It has been concluded that C5b-9 induces trans-activation of receptor tyrosine kinases in association with ERK2 activation, AA release and PGE (2) production in cultured glomerular epithelial cells (GEC) and glomerulonephritis. [Cybulsky et al, Am. J. Pathol. 155:1701(1999)].

Complement activation and membrane assembly of sublytic C5b-9 play an important role in inflammation by promoting cell proliferation and by rescuing cell apoptosis. The Sublytic concentrations of C5b-9 increase Ca⁺ influx, activate phospholipases, increase level of diacylglycerol (DAG) & ceramide, activates protein kinase C (PKC) and generate arachidonic acid. In post-mitotic cells such as oligodendrocytes (OLG) and skeletal muscles, C5b-9 reverses the differentiation of the cell phenotype. [Shirazi et al, J. Neurochem. 48:271(1987)]. Sublytic C5b-9 also induces proto-oncogenes, activates the cell cycle, and enhances survival by inhibiting apoptosis. [Rus et al, J. Immunol. 156:4892(1996); Halperin et al, J. Clin. Invest. 91:1974(1993)]. In OLG loss of differentiation due to C5b-9 attack was associated with the activation of proto-oncogene c-jun, c-fos, and junD and induction of AP1 DNA binding activity. C5b-9 is the most potent ERK1 inducer. ERK1 activation was preceded by activation of membrane-associated Gi, Ras and Raf-1 then activation of cytoplasmic MAPK/ERK kinase (MEK) 1. Trimeric Gi protein was also activated [Niculescu et al, J. Immunol. 158:4405(1997); Niculescu et al, J. Biol. Chem. 269:4417(1994)].

Complement proteins also play a key role in neurodegenerative diseases such as Alzheimer. In several models it has been demonstrated that C5a, via an anti-apoptotic activity provides neuroprotection [Mukherejee et al, J. Neurochem. 77:43 (2001)]. Thus, it is important to specifically block the formation of MAC on the CIC thereby regulating the transfer of MAC in significant doses to avoid the necrosis of the neuronal tissue. The tissue damage by complement and the lytic doses of the MAC leading to tissue necrosis and inflammatory responses has been the key mechanism in diseases such as lung injury, injury to podocytes, cardiomyopathies, myasthenia gravis, multiple sclerosis, cerebral lupus erythematosus, Guilain-Barre syndrome, Alzheimer's disease, lupus nephritis, membranous nephritis, membrane proliferative glomerulonephritis, rheumatoid arthritis, systemic lupus erythematosus, Behcet's syndrome, juvenile idiopathic arthritis, Sjogren's syndrome, atheroma, thyroiditis, infertility, vasculitis, post bypass syndrome, and tissue incompatible transplantation.

### SUMMARY OF THE INVENTION

The invention relates to the formation of Membrane Attack Complex (MAC) [also referred as Terminal Complement Complex (TCC) or C5b-9] on CIC and the applications of measuring complement products including C1q, C3, C4, C5, MAC and its components on CIC as diagnostic markers and reduction in the amount of the MAC on CIC by blocking the formation of MAC on CIC or selectively removing the MAC from the CIC for treatment or prophylaxis of the complement and CIC mediated diseases.

The present invention describes the presence of non-covalently associated complement proteins C1q, C3, C4, C5 and C5b-9 with the CIC. The invention describes for the first time the formation of MAC on the CIC. The experimental data presented in the invention describes that the MAC and components of early complement activation i.e. C1q, C3, C4, C5 and C5b-9 present on CIC are not linked to CIC via covalent linkage. The invention describes that during plasmapheresis the activated complement proteins C1q, C3, C4, C5 and MAC present on CIC are released from the CIC changing the nature of CIC from pathogenic to non-pathogenic. The invention provides methods for measuring the complement activation products i.e C1 q, C3, C4, C5 and MAC on CIC and their usefulness in monitoring the disease activity, and therapy status of complement and CIC mediated diseases including infectious diseases. The invention also provides methods for developing therapies to block the formation of the excessive MAC on CIC to provide beneficial effects in such diseases including autoimmune disorders, cardiovascular disorders, hematological disorders, oncological disorders, kidney diseases.

### BRIEF DESCRIPTION OF DRAWINGS

In the drawings forming the disclosure of this invention:

FIG. 1 illustrates the binding of AHG (aggregated human gamma-globulin) used as immune complex model. A standard curve for binding of AHG was generated in ELISA technique. A linear binding was achieved using the concentration of 2.34, 4.68, 9.37, 18.75, 37.5, 75, 150 and 300 μg/ml of AHG. The appropriate dilutions were made in PBS/Tween 20.

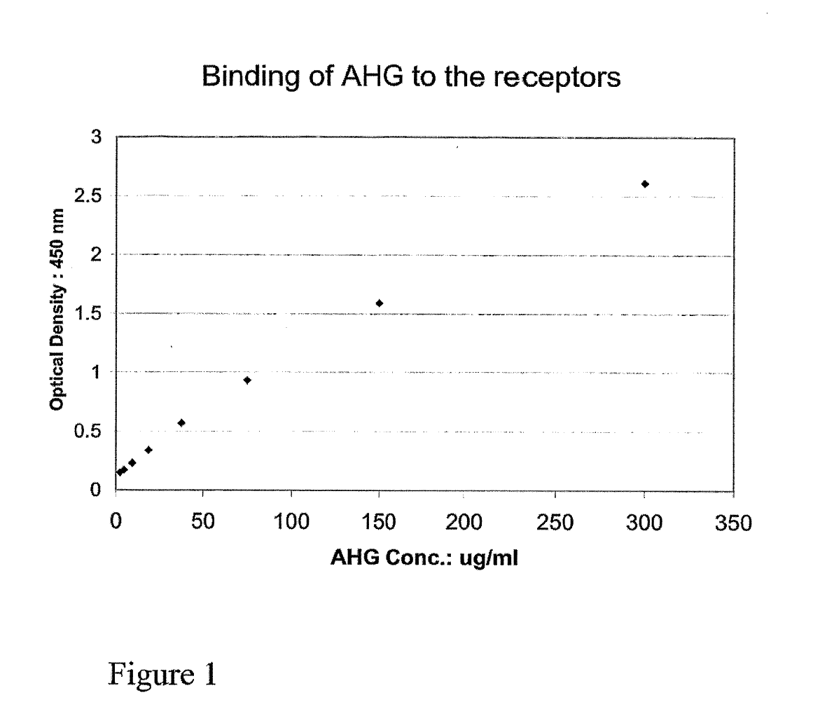

FIG. 2 illustrates the presence of CIC with IgG, IgA and IgM immunoglobulin isotypes in patient's plasma suffering from SLE and RA.

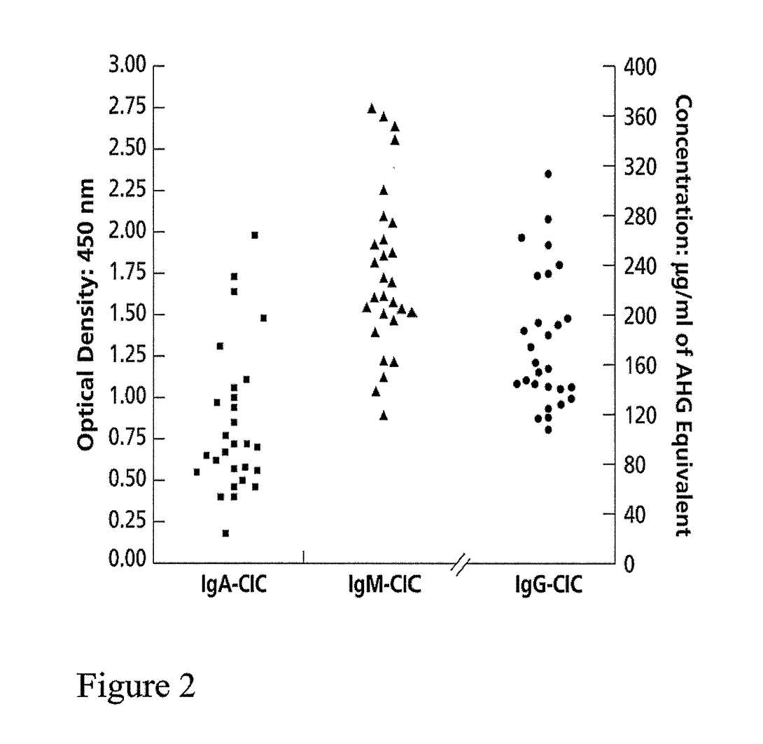

FIG. 3 demonstrate the presence of activated complement components C1q, C3, C4, C5 and C5b-9 in the CIC present bound to CIC in patient plasma from autoimmune diseases.

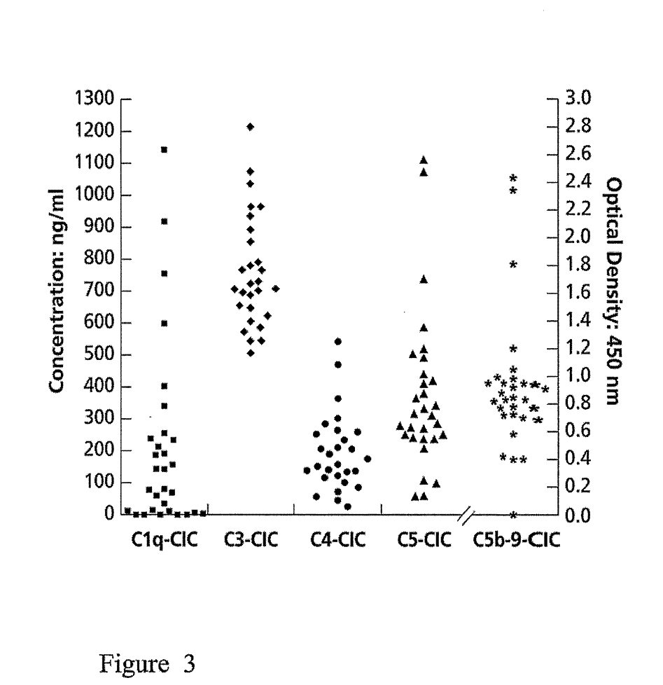

FIG. 4 illustrates the standard curves for (a) C1q, (b) C3, (c) C4, (d) C5 and (e) IgG-CIC used for determining the concentration of the respective components present within the CIC from the patient plasma sample.

FIG. 5 demonstrates the effect of 25 mM EDTA (ethylene diamine tetracetic acid) on the binding of activated complement protein C5 and C5b-9 to CIC. The samples subjected to estimation of C5 and C5b-9 was mixed with appropriate concentration of EDTA so as to bring the final concentration to 25 mM at pH 7.5. Two parallel sets of paired samples from the same patient, one treated with EDTA and other control group without EDTA were subjected to C5 and C5b-9 estimation. As demonstrated in the Figure the treatment of plasma with EDTA decreased the quantities of the complement proteins associated with CIC by several fold. It was concluded from this experiment that the complement proteins in these CIC were not linked with them due to covalent linkage.

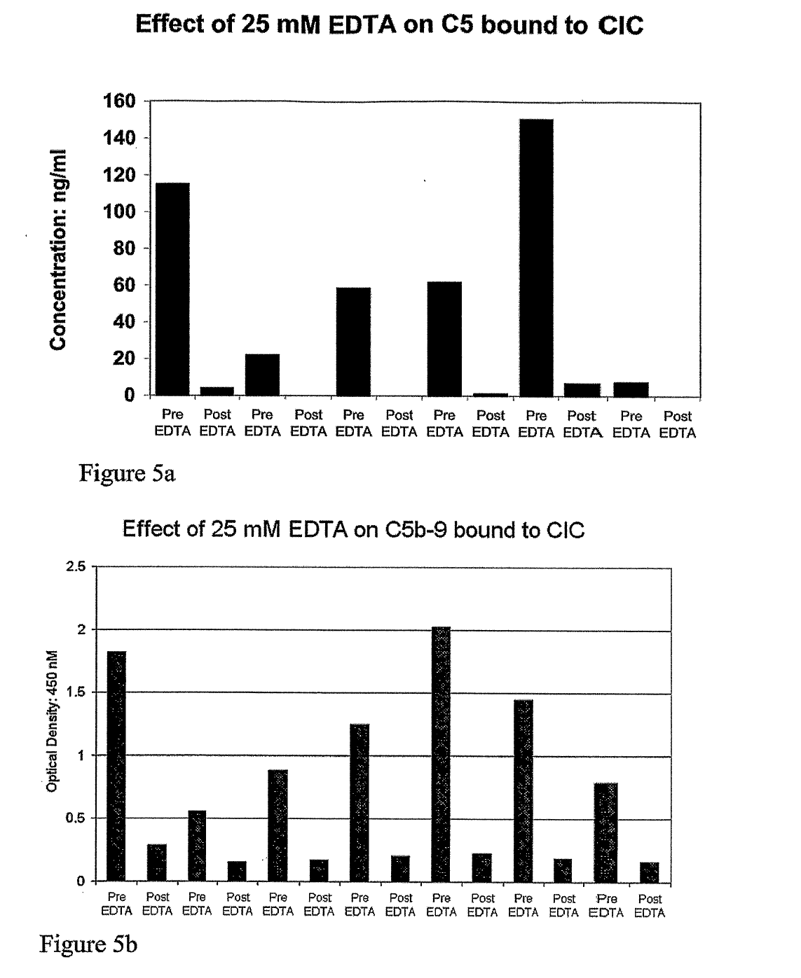

FIG. 6 demonstrates the effect of plasmapheresis on complement binding to CIC. Sequential samples from patient's undergoing kidney and heart transplant and treated with plasmapheresis to achieve beneficial clinical results were obtained for analysis. These samples were then analyzed for the amount of IgG-CIC, IgA-CIC and IgM-CIC (IgG-CIC, CIC composed of IgG, IgA-CIC, CIC composed of IgA, IgM-CIC, CIC composed of IgM). The samples were subjected to measurement of complement proteins C1q, C3, C4, C5 and C5b-9 bound to CIC pre and post plasmapheresis. As demonstrated in FIG. 6, the complement levels bound to CIC decreased significantly with the plasmapheresis. This provided an added experimental proof that in vivo interaction of complement with CIC is not mediated by covalent linkage.

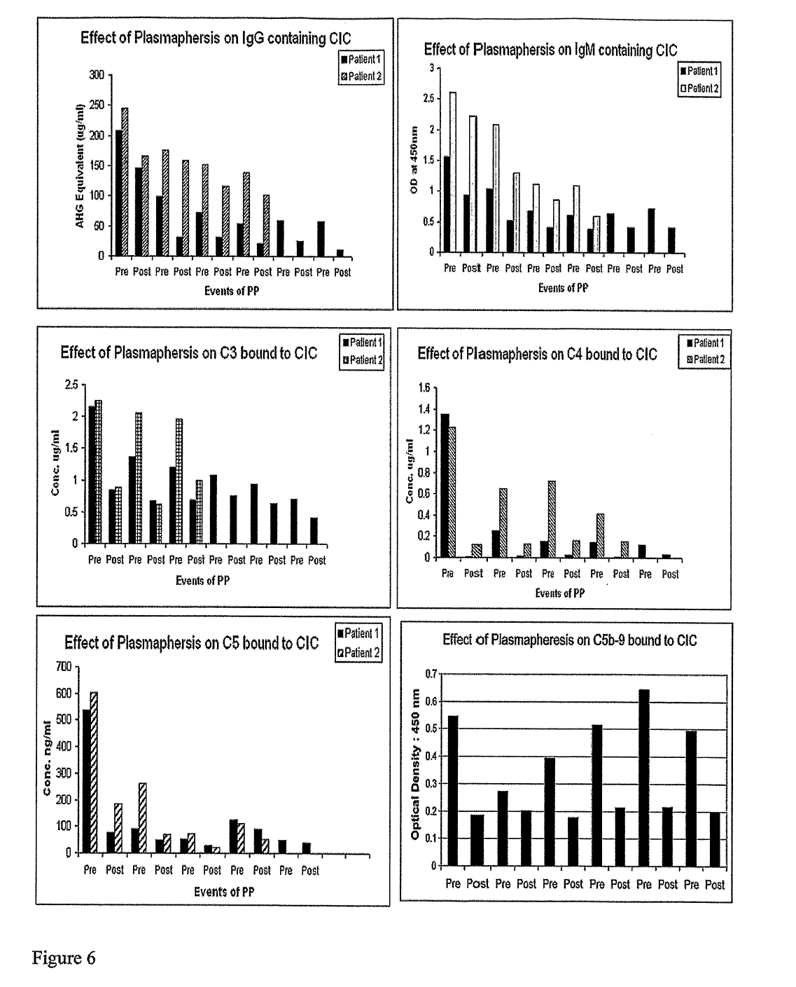

FIG. 7 demonstrates the 2D SDS-PAGE (two dimensional sodium dodecyl sulfate polyacrylamide gel electrophoresis) of CIC purified from a rheumatoid arthritis patient. The CIC were purified using an affinity resin made by coupling the receptor protein isolated as per patent publication (PCT/US02/24301). The receptor protein was coupled to NHS activated Sepharose beads (NHS—Sepharose 4B FF, Pharmacia AB, Piscataway, N.J., US). The resin was packed in a volume of 1.5 ml in a sterile disposable polystyrene column. The patient plasma was allowed to interact with the resin and after washing the non-bound material, the CIC were eluted by lowering the pH to 3.5 with Glycine-HCl buffer. The purified CIC were reduced using DTT and 2ME and the proteins were first separated based on charge in the first dimension using IEF and based on molecular weight on the second dimension by polyacrylamide gel electrophoresis. The CIC components were recognized by comparing the gel image to 2D SDS-PAGE image of human serum proteins in the NBRF protein database. The CIC purified with the affinity column displayed the presence of heavy chain of globulins both γ and μ heavy chains and both light K and λ light chains thereby confirming the identity of CIC. The association of acute phase serum protein CRP was also identified associated in the CIC.

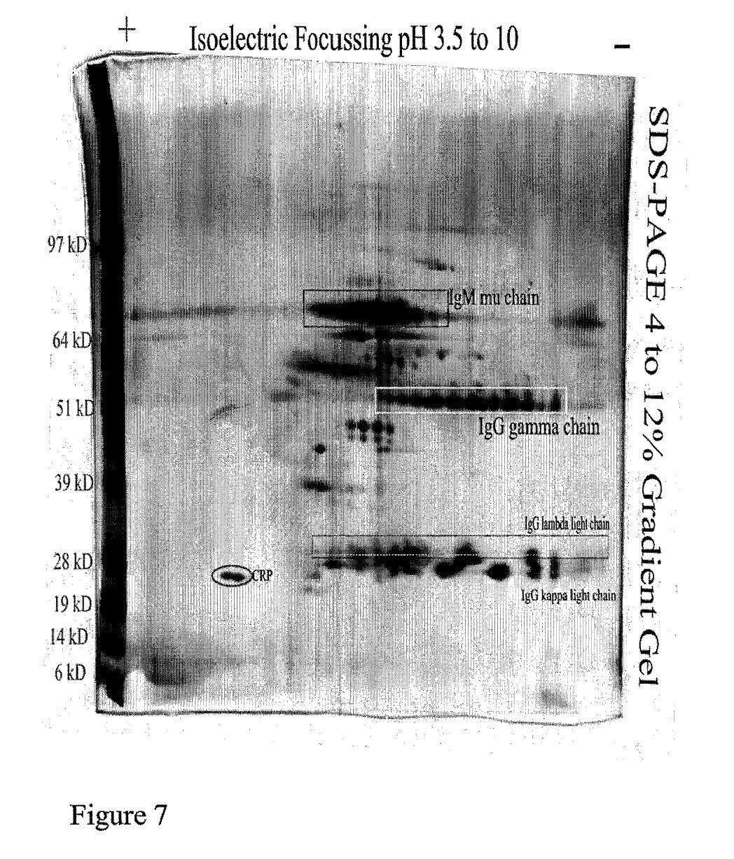

### DETAILED DESCRIPTION

The following terms have the following meaning:
Circulating Immune Complexes (CIC): Antigen antibody complexes that circulation in plasma are referred as circulating immune complexes.
Apoptosis: Programmed cell death. A process of cell death characterized by DNA cleavage, nuclear condensation, and plasma membrane blebbing that leads to phagocytosis of cell without undergoing inflammation.
Necrosis: The sum of morphological changes indicative of cell death and caused by progressive degradative action of enzymes, associated with inflammation.
Innate immunity: Protection against infection that relies on mechanisms that exist before infection, are capable of rapid response to microbes and reacts in essentially same way to repeated infections.
Adoptive immunity: The form of immunity that is mediated by lymphocytes and stimulated by exposure to infectious agent.
Mimotope: An entity that can mimic the antigenic epitope.
Pleitropic: Triggering of multiple function by the same molecule.

As used herein, the term “isolated” means captured in a manner which renders the composition useful for one or more of the diagnostic or therapeutic purposes described herein. Using receptors isolated (prepared as detailed in PCT 20, 010, 801 Chauhan et al, incorporated by reference herein) from cell lines, applicant for the first time has demonstrated the presence of C5 and MAC on CIC. The second important finding reported in the instant application is that complement bound to the CIC is not linked by covalent bond. Thus CIC acts as a substrate for formation of C5B-9 and subsequent transfer to the target cell surface. The presence of higher amounts of C5b-9 on CIC leads to the transfer of lytic doses of C5B-9 to cell surface leading to necrosis, the sublytic doses of C5b-9 lead to cell activation, proliferation, apoptosis and a number of other cellular events. Biochemical or biological molecules restricting the formation of MAC on CIC provides a therapeutic target as the reducing the amount of MAC from lytic doses to sublytic is a beneficial approach for treating complement and CIC mediated injuries.

In the experiments, receptors were isolated from a lymphoblastoid cell line that binds to CIC composed with IgG, IgM and IgA isotypes of immunoglobulin. These complexes are composed of antigen, antibody and other acute phase reactants from the patient plasma such as complement proteins, C reactive protein and serum amyloid protein (SAP). Subsequently, after capturing the CIC on a solid phase coated with the receptor, the CIC was analyzed for their composition. In order to analyze the composition of the CIC from disease patients, ELISA based assays were used. These assays were developed by coating the ELISA plates with the receptor specific for capture of the CIC. The captured CIC on the ELISA plates were probed with secondary antibodies in a two step process with enzyme coupled to antibodies. To demonstrate the presence of complement proteins in the CIC, antibodies directed to complement proteins were specifically used to demonstrate their presence in the CIC. No external additive was added to the interaction, thus no outside interference from other proteins was involved. A previously titrated secondary antibody-HRP conjugate was used to measure the binding of isotype specific CIC. For measuring the complement proteins bound to CIC, the antiserum specific to each complement protein was allowed to interact with the complex bound to the solid phase of the ELISA plates. Subsequently, secondary antibody-HRP conjugate directed to this serum was used for measuring the amount of complement proteins bound within these complexes. Purified proteins and in vitro formed complexes were used as standards in separate wells to quantitate constituents of these CIC.

In our analysis, serum samples from patients with rheumatoid arthritis (RA) and systemic lupus erythematosus (SLE) were used to demonstrate that during disease activity, the CIC evidence significant variation in their composition with respect to the presence of immunoglobulin isotypes IgG, IgA and IgM. In addition to the composition of the CIC isotypes, the sera from these patients also demonstrated significant levels of complement proteins C1q, C3 C4, C5 and C5b-9 associated with the CIC. Opsonization of CIC with these complement proteins can clear apoptotic debris generated during the normal physiology, as well as during an infection. Any defect in clearance of CIC, or the excessive formation of CIC during infection is a major pathologic event resulting in development of auto immune disorders [Walport M J, Arthritis Res. 4(suppl 3):S279 (2002)]. For the first time, however, applicant has demonstrated the presence of the C5 component of complement and the reactivity to C5b-9 bound to the CIC. However using the antibodies directed towards C5a, no reactivity was detected, thus suggesting that C5a, after cleavage from the C5b, falls into the soluble phase and does not form a part of the complex.

Thus, split product of complement C3b and C4b binds to proteins via the formation of a thiol ester bond that gets exposed via activation. The product of the C4A gene binds to CIC by formation of an amide bond, while the product of the C4B gene binds via a carboxyl group through esterification. For the first time applicant demonstrates that the major portion of the activated complement components present on CIC are not covalently attached but only a small portion of the complement appears to be linked via covalent linkage. In patients undergoing plasmapheresis, it was observed by us that post therapy the levels of the complement proteins C1q, C3, C4, C5 and MAC associated with CIC were significantly reduced. It is postulated that the beneficial effect of the plasmapheresis therapy in these patients is mediated due to decrease in the complement proteins associated with the CIC. To confirm this fact we performed two experiments. In one experiment, the effect of EDTA on the complement proteins bound to CIC was analyzed. In this experiment we demonstrated that by treating the serum sample with ethylene diamine tetra acetic acid (EDTA), complement proteins bound to the CIC are released. Serum samples that had been previously tested for the presence of CIC and complement in the presence and absence of EDTA were analyzed. The serum samples from five patients were diluted 1:20 with PBS containing 0.05% Tween 20. One set of samples was treated with 25 mM EDTA that was included in the diluent buffer. The inclusion of EDTA dramatically reduced the binding of complement proteins C3, C4, C5 and C5b-9 in the CIC. Thus, the inclusion of 25 mM EDTA in the PBS as a diluent, and subsequently in the incubation step during the binding, while not affecting the amount of immunoglobulin isotype amounts, eliminated the presence of the complement proteins associated with the CIC. The covalent linkage of the complement to CIC tags these molecules to be cleared by binding with the complement receptor 1 (CR 1) present on the erythrocytes and therefore makes them biologically non-available for signaling functions attributed to the complement. Since the non-covalently associated complement is biologically available it can be utilized for signaling pathways and thus can play key role in the disease pathogenesis.

In another experiment the patient serum samples collected from the pre and post plasmapheresis were analyzed for the presence of complement proteins bound to CIC. In plasmapheresis therapy the one fifth of the plasma volume from patient was replaced with the 5% human serum albumin using the extracorporeal circuit. The experiment demonstrated that plasmapheresis affects the binding of complement proteins to CIC. A dramatic drop from 40 to 90% was demonstrated for various complement proteins which bind to the CIC. Accordingly, the beneficial effect of the therapy was provided.

The nature of the CIC of the samples was established by selectively isolating CIC from patient with RA and SLE. The CIC was purified using an affinity resin developed by coupling the receptor preparation with Sepharose FF 4B (Pharmacia, Piscataway N.J.). The affinity resin was then utilized to capture CIC from the RA and SLE patients. The individual components of CIC were displayed on SDS-PAGE (sodium dodecyl sulfate polyacrylamide gel electrophoresis) and the individual components were recognized by Western Blotting. The identity of individual immunoglobulin chains was established using μ and γ heavy chain specific antibodies. The identity of the components of the CIC was also established by subjecting the CIC to 2D SDS-PAGE analysis. Using the comparative analysis of the results from the NBRF protein database, the presence of IgM and IgG heavy chains as well as both kappa and lambda light chains in the captured CIC was established.

The measurement of CIC, composed with various immunoglobulin isotypes and the activated complement component C1q, C2, C3, C4, C5, and C5b-9 bound to CIC, provides a useful indicator as one aspect of the invention. In this aspect, the modulation of the complement on CIC by an appropriate chemical, biochemical, peptide or biological is useful in the treatment of disease, including: Renal Diseases Anti-Glomerular Basement Membrane Disease Renal Vasculitis: Focal Necrotizing GlomerulonephritisoRapidly progressive glomerulonephritisoWegener”s granulomatosus (WG)oMicroscopic polyangitisoldiopathic RPGN Focal Segmental Glomerulosclerosis Systemic Lupus Erythematosus Anti-Glomerular Basement Membrane Disease Neurological Disease Eaton-Lambert Syndrome Guillain-Barre” syndrome Amyotrophic Lateral Sclerosis Myasthenia Gravis Inflammatory Polyneuropathy Multiple Sclerosis Alzheimer Hematological Disease Mycelia and Cryoglobulinemia Thrombotic Thrombocytopenic Purpura Idiopathic Thrombocytopenic Purpura Allaoantibodies in Hematologic Disease Rheumatologic Disease Rheumatoid Arthritis Rheumatoid Vasculitis Scleroderma Dermator Early stages of Scleroderma Dermatomyosistis Polymyosistis Sjogren”s syndrome Behcet”s disease Other Disease Pemphigus Vulguris associated to antibodies to squamous epithelium Bullous pemphigoid associated to antibodies to dermal basement Cardiovascular Disease: Myocardial Infarction Cardiomyopathies Ischemia reperfusion injury Transplant Neoplastic Diseases

### EXAMPLES

### Example One

The example describes the development and use of ELISA based assays for measurement of components of CIC i.e. Antibody isotypes, IgG (IgG1, IgG2, IgG3, IgG4), IgA, IgM; complement proteins, C1q, C3, C4, C5 and C5b-9; other acute phase proteins associated within the CIC.

Details on performing the assay for measuring the components of CIC: (1) Purified receptor preparation the proteins binding specifically to CIC via Fc portion were dissolved in an alkaline buffer (0.1M sodium carbonate pH 9.6).

(2) The alkaline solution with receptor preparation was placed in contact with the plate at 4° C. for 12 to 24 hours.

(3) The coating was removed form the plate and plate washed three times with a solution of sodium chloride (0.15M), buffered by sodium and potassium phosphate (0.01M, pH 7.2 to 7.4) (PBS) to remove unbound receptors.

(4) Thereafter to block free sites the plate was placed in contact with 100 μl of 1% BSA dissolved in PBS containing 0.05% Tween-20.

(5) The blocking solution was removed and plates washed three times with PBS fortified with 0.05% of Tween-20 (v/v).

(6) The sera from patients were diluted properly with a solution of PBS prior to testing. In this example we diluted the sera 10 volumes and 20 volumes of PBS.

(7) A total of 0.1 ml aliquots of diluted sera were placed into appropriately designated wells. For this example duplicate determinations were performed for each specimen and average values were used for calculations.

(8) The plates were kept at 37° C. in humid container for two hours.

(9) The plates were washed again with PBS/Tween-20.

(10) The plates were filled with 100 μl of appropriate anti serum for measurements (anti-human IgG-HRP, anti-human IgM-HRP, anti-human IgA-HRP, anti-human C3, anti-human C4, anti-human C1q, anti-human C5 and anti-human C5b-9). The plates were incubated at room temperature for sixty minutes.

(11) The plates were washed again and the plates that received the HRP conjugates were developed for HRP enzyme activity. Otherwise the wells in plates without conjugated antiserum were filled species, specific anti-HRP conjugate and further incubated for sixty minutes at room temperature.

(12) After washing each plate well in the plate was assayed for horseradish peroxidase activity by addition of 100 μl substrate buffer (TMB substrate).

(13) The reaction was monitored for the development of color and at appropriate color density the reaction was terminated by addition of 25 μl of 2.5 M H₂SO₄.

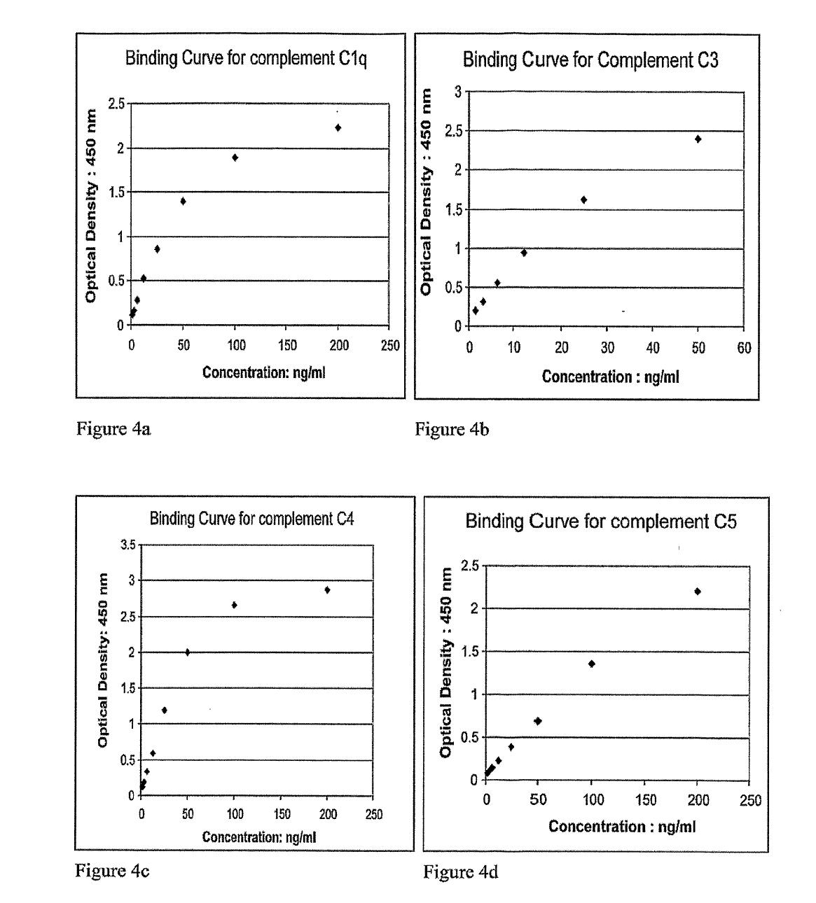

(14) Optical Density was measured and plotted against the standards concentrations and the linear equation was used to obtain the quantity of components in the CIC (FIG. 4).

### Example Two

### Purification and Analysis of CIC on 2D SDS-PAGE:

(1) The receptor protein binding was conjugated to NHS-activated (n-hydroxyl succinamide) sepharose 4 B (Pharmacia, Piscataway, USA).

(2) The free sites on the resin were blocked with excess of 1M Tris-HCl pH 7.5.

(3) The resin was washed with PBS to remove unbound Tris-HCl.

(4) In a column with one ml of resin a total of 1.5 ml of patient plasma was placed in contact with the receptor bound resin.

(5) The plasma was allowed to flow under gravity and fifteen times PBS was allowed to flow into the column to remove unbound plasma proteins.

(6) The bound CIC were eluted with low pH buffer (Glycine-HCl, 0.1M, pH 3.5).

(7) Captured CIC were concentrated to a final volume of 300 μl.

(8) Twenty micro-liters of the purified CIC proteins were mixed with IEF renaturing solution consisting of 8M Urea, Bridge 58, NP40, 2ME, β-Octylglucoside.

(9) The sample was mixed properly and applied to 7 mm IPG strips (Bio-Rad, Hercules, Calif.). The strips were left for 16 hours at room temperature thereafter the IPG strips were subjected to isoelectric focusing on pH 3.5 to 10 IPG strip in accordance with manufacturer recommendation.

(10) A total amount of 10,000 Volt-hours were applied during the IEF.

(11) After the isoelectric focusing the strips were removed from the IEF cell and drained of excess mineral oil and incubated with buffer containing 8M Urea, 0.375 M of Tris-HCl buffer pH 8.8, 20% Glycerol, 100 mM DTT for 15 minute with constant shaking.

(12) After the first incubation the IEF strips were incubated for another fifteen minute at room temperature in a buffer with composition similar to earlier buffer containing 125 mg of Iodoacteamide per 10 ml of buffer.

(13) Thereafter the IPG strips were overlaid for second dimension run on 4 to 12% SDS-PAGE NuPAGE gel (Invitrogen, Carlsbad, Calif.).

(14) The electrophoresis was carried out in MOPS buffer at 170 volts for two hours. The gels were then fixed in acetic acid and ethanol fixative.

(15) The gels were stained with silver stain. The comparative analysis of the, CIC were done utilizing the 2D protein database from EMBO to establish the identity of globulin heavy and light chains.

It is to be understood that the present invention has been described in detail by way of illustration and example in order to acquaint others skilled in the art with the invention, its principles, and its practical application. Further, the specific embodiments of the present invention as set forth are not intended as being exhaustive or limiting of the invention, and that many alternatives, modifications, and variations will be apparent to those skilled in the art in light of the foregoing examples and detailed description. Accordingly, this invention is intended to embrace all such alternatives, modifications, and variations that fall within the spirit and scope of the following claims. While some of the examples and descriptions above include some conclusions about the way the invention may function, the inventor does not intend to be bound by those conclusions and functions, but puts them forth only as possible explanations.

All references cited herein are hereby incorporated by reference as though fully set forth in the application

What is claimed is:

1. An isolated complex comprising one or both of complement activation product C5, and membrane attack complex (C5b-9) associated with circulating immune complex.

2. A method for inhibiting the formation of a non-covalent combination of membrane attack complex and circulating immune complex comprising application of an inhibitor selected from the group consisting of a monoclonal antibody, peptide mimotope, or small molecule in patients suffering from complement and circulating immune complex mediated diseases.

3. A method for screening candidate compositions or processes for an ability for inhibiting the formation of membrane attack complex on circulating immune complex comprising assessing the composition or process for the reduction in membrane attack complex associated with circulating immune complex as a result of the application of the candidate composition or process.

4. A method of monitoring the formation of membrane attack complex and other split products of C5 on circulating immune complex from the serum, plasma, cerebrospinal fluid and other bodily fluids in diseases associated with complement and circulating immune complex pathogenesis comprising measuring the formation of said products and assessing for symptoms of said disease.

5. Isolated complexes comprising one or more of the group consisting of non-covalent linked complement split products C1q, C3, C4, C5 and membrane attack complex on circulating immune complex.

6. A method of inhibiting non-covalent association of C1q, C3, C4, C5 and membrane attack complex to circulating immune complex comprising application of an inhibitor selected from the group consisting of a monoclonal antibody, peptide mimotope, or small molecule in patients suffering from complement and circulating immune complex mediated diseases.

7-8. (canceled)

9. A process for quantitative measurement for the presence of complement C5 and C5b-9 associated with circulating immune complex, the process comprising the following steps:
a. Providing a test device comprising a receptor preparation in solid phase as a capture reagent for circulating immune complex;
b. Establishing a selected working range for an immunoassay within said ranges of composition of circulating immune complex, IgG-CIC 2 to 1000 μg/ml, IgA-CIC 0 to 1000 μg/ml, IgM-CIC 0 to μg/ml, C1q bound to CIC 0 to 10 μg/ml, C3 bound to CIC 0 to 30 μg/ml, C4 bound to CIC 0 to 10 μg/ml, C5 bound to CIC 0 to 10 μg/ml and C5b-9 0 to 10 μg/ml;
c. Constructing a standard assay curve by plotting relative degree of immunochemical binding of said circulating immune complex components to the test device;
d. Interacting a fixed concentration of immunospecific conjugate of said substances, the composition of complexes resulting from said immunological substances and immunospecific conjugate being within the selected working range limits;
e. Providing a test system comprising of said test device, said immunospecific conjugate, said immunological substances, the amount of said immunospecific conjugate being substantially equivalent to said fixed concentration of immunospecific conjugate, and the amount of said immunospecifically determinable substance being appropriate to produce a known degree of immunochemical binding corresponding to a predetermined point on said standard curve, thereby enabling quantitative assaying of one or more of complement proteins C1q, C3, C4, C5 and C5b-9 present on circulating immune complex.

10. (canceled)

11. A process for measurement of one or more complement proteins C1q, C3, C4, C5 and C5b-9 from plasma or other bodily fluids of animals suffering from or at risk of suffering from a disease or condition, including but not limited to autoimmune, cardiovascular, neurodegenerative disorders, oncological diseases and infectious disease, said process comprising:
a. Providing a test device comprising a receptor preparation in solid phase;
b. Establishing selected working ranges for said immunoassay within said ranges for complement proteins;
c. Constructing a standard assay curve by plotting relative degree of immunochemical binding of said complement component(s) to the test device;
d. Interacting a fixed concentration of a immunospecific conjugate directed to complement proteins and immunospecific conjugate being within pre selected working range limits;
e. Providing a test system comprising of said test device, said immunospecific conjugate, said immunological substances, the amount of said immunospecific conjugate being substantially equivalent to said fixed concentration of immunospecific conjugate, and the amount of said immunospecifically determinable substance being appropriate to produce a known degree of immunochemical binding corresponding to a predetermined point on said standard curve, thereby enabling quantitative assaying of one or more of complement C1q, C3, C4, C5 and C5b-9 present on circulating immune complex.

12. A process for quantitation of immunoglobulin isotype composition of circulating immune complex or antigens bound within circulating immune complex comprising using an ELISA based on receptor based capture mechanism, said process comprising:
a. Placing the receptor on solid phase of ELISA plates, micro beads or other suitable surface;
b. Attaching the biotin or other form of detection tag on the antigen or antibody;
c. Mixing the tagged antigen or antibody with the patient plasma, patient serum, sinuovial fluid, cerebrospinal fluid (CSF) or other bodily fluid;
d. Placing the mixture in contact with receptor attached to the solid surface;
e. Washing the unbound components with buffers;
f. Quantitating the tagged antigen or antibody with a reagent including, but not limited to Avidin-Horse Radish Peroxidase and color development reagents.

13. A process as set forth in claim 3 for screening the composition of a blocking agent for the formation of membrane attack complex and deposition of C5 on circulating immune complex.

14. (canceled)

15. A process for screening a composition that targets blocking of complement activation or other component assembly in the circulating immune complex as set forth in claims 11, and modulating the binding of serum acute phase proteins bound to circulating immune complex, said process comprising:
a. Attaching the receptor to solid phase or studying the interaction in the liquid phase, allowing the interaction of the circulating immune complex with the receptor in presence of complement proteins to activate complement deposition or other acute phase proteins on circulating immune complex;
b. Placing the blocking composition during the activation of the complement on circulating immune complex or association of serum acute phase protein;
c. The composition being selected from the group consisting of a chemical, biochemical, protein, peptide and monoclonal;
d. Obtaining initial data indicating whether the formation of membrane attack complex and binding of complement C1q, C2, C3, C4, and C5 is inhibited on the circulating immune complex;
e. Obtaining data indicating whether the serum acute phase proteins associated with the circulating immune complex is inhibited.

16-17. (canceled)

18. A method of reducing disease symptoms in an individual comprising: identifying an individual in need of reducing the symptoms due to increased complement fixation on circulating immune complex leading to inflammation and tissue necrosis by administering a composition comprising a monoclonal antibody, peptide, mimotope or active molecule

19. A process in accordance with claim 11 that further comprises contacting a receptor during interaction with circulating immune complex and complement with at least one of a humanized monoclonal antibodies, active molecules, peptides and mimotopes and obtaining data indicative of whether the activation of complement has been inhibited.

20. A process in accordance with claim 17 that further comprises inoculating patients or animals with the immune complex and composition, wherein the immune complex mediated immune responses are altered providing beneficial effect.

21. The method of claim 2 wherein the disease is selected from the group consisting of systemic lupus erythematosus, rheumatoid arthritis, cardiovascular diseases, kidney diseases, and autoimmune diseases.

22. The method of claim 4 wherein the disease is selected from the group consisting of autoimmune diseases, cardiovascular diseases, neurodegenerative diseases, infectious disease and oncological diseases.

23. The method of claim 6 wherein the disease is selected from the group consisting of systemic lupus erythematosus, rheumatoid arthritis, cardiovascular diseases, kidney diseases, and autoimmune diseases.
